(19) United States
(12) Patent Application Publication
(10) Pub. No.: US 2013/0046222 A1
(43) Pub. Date: Feb. 21, 2013

(54) APPARATUS FOR AN ADJUSTABLE CUSTOM MOLDED ARCH SUSPENSION SYSTEM

(71) Applicant: CHRISTOPHER M. GIZZI, Brooklyn, NY (US)

(72) Inventor: CHRISTOPHER M. GIZZI, Brooklyn, NY (US)

(21) Appl. No.: 13/568,178

(22) Filed: Aug. 7, 2012

Related U.S. Application Data
(60) Provisional application No. 61/525,039, filed on Aug. 18, 2011.
(60) Provisional application No. 61/617,265, filed on Mar. 29, 2012.

(51) Int. Cl. A61F 5/00

(52) U.S. Cl. 602/28

(57) ABSTRACT

The invention relates to a custom molded ankle foot orthosis having a cam buckle operated adjustable arch suspension system positioned therein. The custom molded ankle foot orthosis having an inner shell having a first material composition and flexibility, and shaped to accommodate receiving therein and supportingly couple with the foot, and an outer shell having a second material composition, the outer shell shaped to accommodate receiving therein the inner shell. Preferably, the ankle foot orthosis comprises a custom molded stirrup or foot support, at least one mounting or securing strap, an arch support structure, and an arch suspension system. The custom molded stirrup or foot support is arranged for securement to the heel and the lateral and medial sides of a wearer's leg.

### CROSS-REFERENCE TO RELATED APPLICATIONS

This application claims priority to U.S. provisional application Ser. No. 61/525,039, filed on Aug. 18, 2011, and U.S. provisional application Ser. No. 61/617,265 filed Mar. 29, 2012, the contents of each of which is hereby incorporated by reference in its entirety.

### BACKGROUND OF THE INVENTION

1. Field of the Invention

The present invention relates to a custom molded ankle foot orthosis. More particularly, the present invention provides a custom molded ankle foot orthosis having a cam buckle operated arch suspension system for applying a lifting force to suspend or support the talonavicular joint for full control of the hind foot/ankle complex.

2. Description of the Related Art

Numerous ankle braces or supports are known in the art, and have been disclosed in the patent literature for applying a lifting force to one or more portions of the foot. It is well recognized among experts in podiatric and lower extremity biomechanics that the critical pivotal joint of the human foot is the talonavicular joint. Previously, most foot orthotic interventions were designed to control the subtalar joint. Attempts to control the talonavicular joint (medial flange, sustentaculum support) have caused potential irritation to this sensitive part of the human foot. Yet, recent experimental evidence, using arthrodesis procedures of cadaver models, have clearly shown that control of the talonavicular joint will have profound control and limitation of the entire rearfoot complex, including the subtalar and calcaneal-cuboid joints.

During pronation of a foot there are three significant segments of the foot that must be controlled. The heel area during pronation tends to shift into eversion. Eversion is an anatomical condition where the heel, with respect to the ankle, is repositioned and rotates about a longitudinal axis laterally outwardly. The longitudinal arch must maintain a proper biomechanical position and alignment. During pronation the arch moves medially and distally to a flat position, more so in the medial direction. Finally, the forefoot will shift laterally outwardly to abduction. Therefore, all three of these occurrences happen in conjunction and the heel and the arch in the forefoot will shift commensurate with the misalignment of each general foot region.

It should be noted that during collapse of the mid foot longitudinal arch, the skin surface of the heel will remain substantially intact with the weight-bearing surface, but the upper portion of the heel will move laterally inwardly, rotating about a substantially longitudinally extending axis.

To control the talonavicular joint, a force, or moment must be applied medial to the axis of the entire rearfoot complex, which is slightly deviated in a more vertical alignment to the subtalar joint axis. To prevent plantar and medial migration of the head of the talus, the force must be applied in a dorsal, posterior and lateral direction which would cross almost perpendicular to the axis of rotation of the rearfoot complex. Heretofore no ankle brace has achieved that end.

Therefore, an effective orthotic or orthosis device must address all three of these simultaneously while providing for movement and general athletic motions of the patient. The device should address these misalignment issues and be comfortable and wearable by the patient.

In particular, the spring ligament complex of the human foot is the only structure that is anatomically designed to provide precise support of the talonavicular joint. In severe foot pathologies such as the adult acquired flatfoot secondary to posterior tibial tendon dysfunction, the spring ligament complex becomes attenuated and the resultant foot deformity is uncontrolled with standard foot orthoses. Known in-shoe foot orthoses cannot apply significant moment of force in the proper direction to adequately control the talonavicular joint.

Orthotics and lower limb orthosis devices, in one form, are made for patients by practitioners for a custom fit to accommodate a patients lower limb support needs. It has been found, in the practice of forming foot orthotics or orthoses, that the logistics of transporting product from the factory to the practitioner is time-consuming, as the practitioner must first send a mold back to the manufacturing facility. Also, there is an issue of maintaining product at the practitioner's location whereby constantly sending molds to the manufacturer can deplete the practitioner's supply of stock mold materials. Therefore, in one form it is desirable to have an embodiment where a semi-rigid device used to make a negative mold of a patient's foot is not sent back to the orthosis manufacturer, but rather, can be stored at the practitioner's location for reuse. Further, storage space is generally not plentiful at a practitioner's business location, and maintaining inventory of foot molding products can be very challenging. In one form, the embodiments below disclose a convenient method of storing and stacking the flexible members.

Furthermore, foot orthoses are problematic in that they have not often been comfortable to patients. Therefore, the challenge has been to provide a comfortable off-the-shelf foot orthosis that provides support and adapts to various patients' feet without specific molding. Pre-made inserts have been problematic because of the instability associated with them.

Accordingly, there is a need for an improved ankle foot orthosis that provides the wearer or health care practitioner the ability to control the amount of support or correction they require for the particular diagnosis. Furthermore, there is a recognized need for an improved ankle foot orthosis for the treatment of severe pronation, mild charcot arthopathy, sinus tarsi, ankle arthritis, ankle instability, adult acquired flat foot due to posterior tibial tendon dysfunction (PTTD) (i.e., both stage II & stage III), off-loading of mid-foot plantar ulcerations, and other clinical indications.

### SUMMARY OF THE INVENTION

This invention relates to a custom molded ankle foot orthosis having a cam buckle operated adjustable arch suspension system. In a preferred embodiment, the ankle foot orthosis comprises a custom molded stirrup or foot support, at least one mounting or securing strap, an arch support structure, and an arch suspension system. The custom molded stirrup or foot support is arranged for securement to heal and the lateral and medial sides of a wearer's leg. The stirrup or foot support is designed to receive the wearer's foot and comprises a bottom portion shaped to receive the wearer's heel, and medial side, lateral side and back side portions contiguous from the bottom portion.

The one piece custom molded stirrup or foot support further contains an aperture through which the arch suspension system travels to a cam buckle adjustment system attached to the proximal end of the arch suspension system. Preferably, the arch suspension system is permanently fixed on the opposite side of the cam buckle (i.e., the lateral side) below the patient's maleolous. Optionally, the custom molded arch suspension system can be used on the medial or lateral side of the device to support or suspend the medial longitudinal arch or the lateral longitudinal arch respectively. The custom molded stirrup or foot support is preferably secured to the patient's leg by two fastening devices, preferably hook and loop straps, one on the proximal end of the device and one just above the maleoli.

In accordance with one aspect of this invention, the arch suspension system comprises a lifting pad that includes a first portion secured to the inner lateral side of the bottom portion of the molded stirrup or foot support and is shaped to receive the heel portion of the wearer's foot. Such lifting pad extends upward on the inner medial side of the molded stirrup or foot support to an aperture on the middle of the medial side of the molded stirrup or foot support such that the lifting pad may be attached to the cam buckle (or other adjustment mechanism) positioned on the outside of the molded stirrup or foot support near its proximal end. The medial lifting pad may optionally be releasably secured to the lateral side of the molded stirrup or foot support.

The invention relates to a custom molded ankle foot orthosis having an adjustable arch suspension system, said apparatus comprising: a molded stirrup having lateral, medial and bottom portions, said stirrup configured to receive the foot of a wearer and comprising a bottom portion having a heel receiving end, a contiguous medial portion, and a contiguous lateral portion, said medial and lateral portions extending upward from said bottom portion adjacent said heel receiving end; a medial lifting pad positioned on the inner side of said stirrup, said lifting pad fixedly secured on a first end to an inner lateral side of said stirrup and secured on a second end to a first end of a strap, said strap being positioned through an aperture on said medial portion of said stirrup and adjustably connected at a second end to an adjustment mechanism; and at least two mounting straps arranged to secure the upper and lower lateral and medial portions of said stirrup to a wearer's leg; wherein said adjustment mechanism engages said second end of said strap and applies a lifting force to said medial lifting pad at a wearer's medial arch joint.

Optionally, the custom molded ankle foot orthosis further provides an adjustable amount of lifting force provided by said lifting pad; comprises mounting straps that are hook and loop type fasteners; wherein the adjustment mechanism is a cam buckle; wherein the can buckle comprises: a frame having a pair of side walls with front and rear ends, a loop pivotally attached to said side walls, the loop being adapted for holding one end of said strip of web-like material, a first bar extending between said side walls and attached thereto at a position forwardly of the pivotable attachment of the loop, a second bar mounted on the side walls and extending generally parallel to the first bar, the second bar being adapted for holding said second end of said strap, and said buckle being movable between a locked and an unlocked position.

Moreover, the custom molded ankle foot orthosis optionally comprises outer material of said stirrup that is made of plastic, vinyl, composite, ethylene, polyethylene based plastic and/or a polymer. The inner side of said stirrup optionally includes a cushioning material, and has a thickness between 0.05 inches and 0.10 inches and is configured for use within a wearer's shoe. Furthermore, the ankle foot orthosis is optionally washable, X-ray transparent, and design to treat clinical indications selected from the consisting of severe pronation, mild charcot arthopathy, sinus tarsi, ankle arthritis, ankle instability, adult acquired flat foot due to posterior tibial tendon dysfunction (PTTD) (Stage II & Stage III), and off-loading of mid-foot plantar ulcerations.

It is an object of the present invention to provide a custom molded (i.e., to a patient model) ankle foot orthosis having adjustable custom molded arch suspension system that can be individually adjusted to support or suspend the talonavicular joint for full control of the hind foot/ankle complex. In the preferred embodiment, the suspension system is operated via a cam buckle system. Such a system affords the wearer and the healthcare professional the ability to control the amount of support or correction they require for their particular diagnosis. Some such clinical indications include: severe pronation, mild charcot arthopathy, sinus tarsi, ankle arthritis, ankle instability, adult acquired flat foot due to posterior tibial tendon dysfunction (PTTD) (both Stage II & Stage III), off-loading of mid-foot plantar ulcerations, etc.

It is another object of the invention to provide an ankle foot orthosis having adjustable custom molded arch suspension system that is configured to wear or be usable inside a shoe.

It is still another object of the invention to provide an ankle foot orthosis having adjustable custom molded arch suspension system having a locking back up mechanism.

It is yet another object of the invention to provide an ankle foot orthosis having adjustable custom molded arch suspension system that is washable.

It is another object of the invention to provide an ankle foot orthosis having adjustable custom molded arch suspension system that is made of thermal fused plastic, or any other rigid, non-metallic material.

It is still another object of the invention to provide an ankle foot orthosis having adjustable custom molded arch suspension system that is X-ray transparent for security ease, e.g., such that it need not be removed when passing though security stations.

It is yet another object of the invention to provide an ankle foot orthosis having adjustable custom molded arch suspension system that provides support under the wearer's foot with two straps.

The above and other aspects, features and advantages of the present invention will become apparent from the following description read in conjunction with the accompanying drawings, in which like reference numerals designate the same elements.

### BRIEF DESCRIPTION OF THE DRAWINGS

A further understanding of the present invention can be obtained by reference to a preferred embodiment set forth in the illustrations of the accompanying drawings. Although the illustrated preferred embodiment is merely exemplary of methods, structures and compositions for carrying out the present invention, both the organization and method of the invention, in general, together with further objectives and advantages thereof, may be more easily understood by reference to the drawings and the following description. The drawings are not intended to limit the scope of this invention, which is set forth with particularity in the claims as appended or as subsequently amended, but merely to clarify and exemplify the invention.

For a more complete understanding of the present invention, reference is now made to the following drawings in which:

FIG. 1 shows a lateral side view of the molded ankle foot orthosis showing one preferred exemplary embodiment of the invention constructed in accordance with this invention having two securing straps for securing the orthosis to the ankle and lower limb of the wearer;

FIG. 2 shows an exploded medial side perspective view of the molded ankle foot orthosis showing one preferred exemplary embodiment of the invention constructed in accordance with the present invention having two securing straps for securing the orthosis to the ankle and lower limb of the wearer and an adjustable cam buckle for selectively applying a lifting force to respective portions of the wearer's foot;

FIG. 3 shows an exploded side perspective view of the adjustable custom molded arch suspension system having a lifting pad insert for the molded ankle foot orthosis shown in FIG. 2;

FIG. 4 shows an partially exposed perspective view of the molded ankle foot orthosis secured to the lower limb of a user in accordance with the preferred embodiment of the present invention, further depicting the lifting pad coupled to the cam buckle for selectively applying a lifting force to respective portions of the wearer's foot;

FIG. 5 shows a preferred embodiment of the cam buckle for operating the adjustable custom molded arch suspension system in accordance with the invention;

FIG. 6 shows a cross-sectional view of the molded ankle foot orthosis along line 6-6 as shown in FIG. 4; and

FIG. 7 shows a cross-sectional view of the molded ankle foot orthosis along line 7-7 as shown in FIG. 4.

### DETAILED DESCRIPTION OF THE PREFERRED EMBODIMENTS

As required, a detailed illustrative embodiment of the present invention is disclosed herein. However, techniques, systems, compositions and operating structures in accordance with the present invention may be embodied in a wide variety of sizes, shapes, forms and modes, some of which may be quite different from those in the disclosed embodiment. Consequently, the specific structural and functional details disclosed herein are merely representative, yet in that regard, they are deemed to afford the best embodiment for purposes of disclosure and to provide a basis for the claims herein which define the scope of the present invention.

Reference will now be made in detail to several embodiments of the invention that are illustrated in the accompanying drawings. Wherever possible, same or similar reference numerals are used in the drawings and the description to refer to the same or like parts or steps. The drawings are in simplified form and are not to precise scale. For purposes of convenience and clarity only, directional terms, such as top, bottom, up, down, over, above, below, etc., or motional terms, such as forward, back, sideways, transverse, etc. may be used with respect to the drawings. These and similar directional terms should not be construed to limit the scope of the invention in any manner.

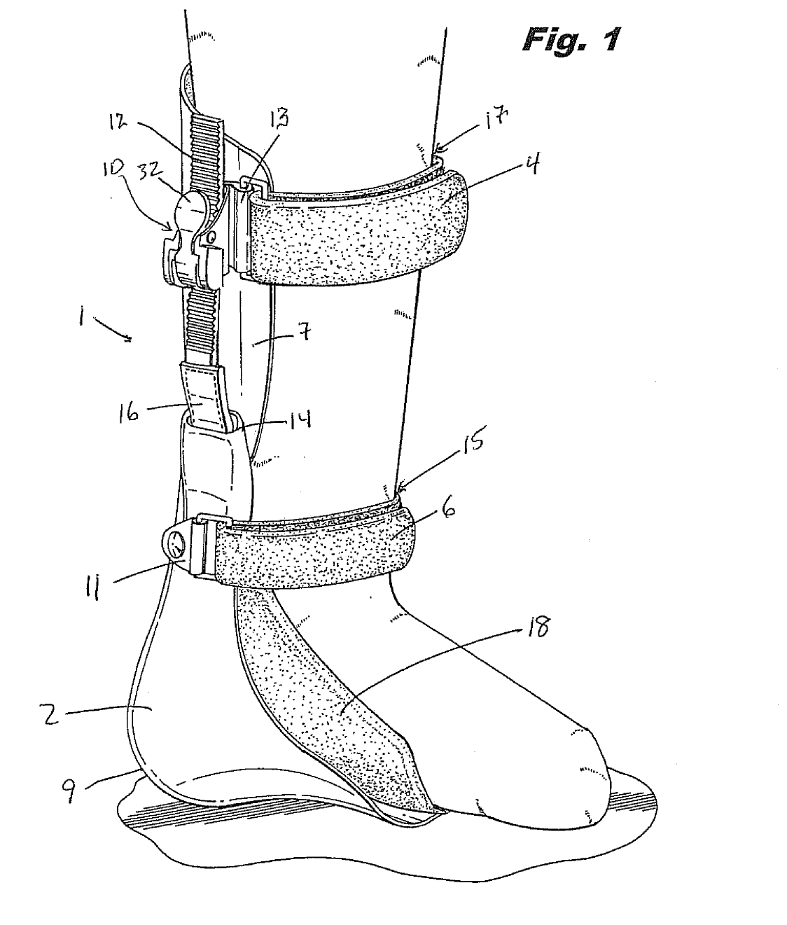

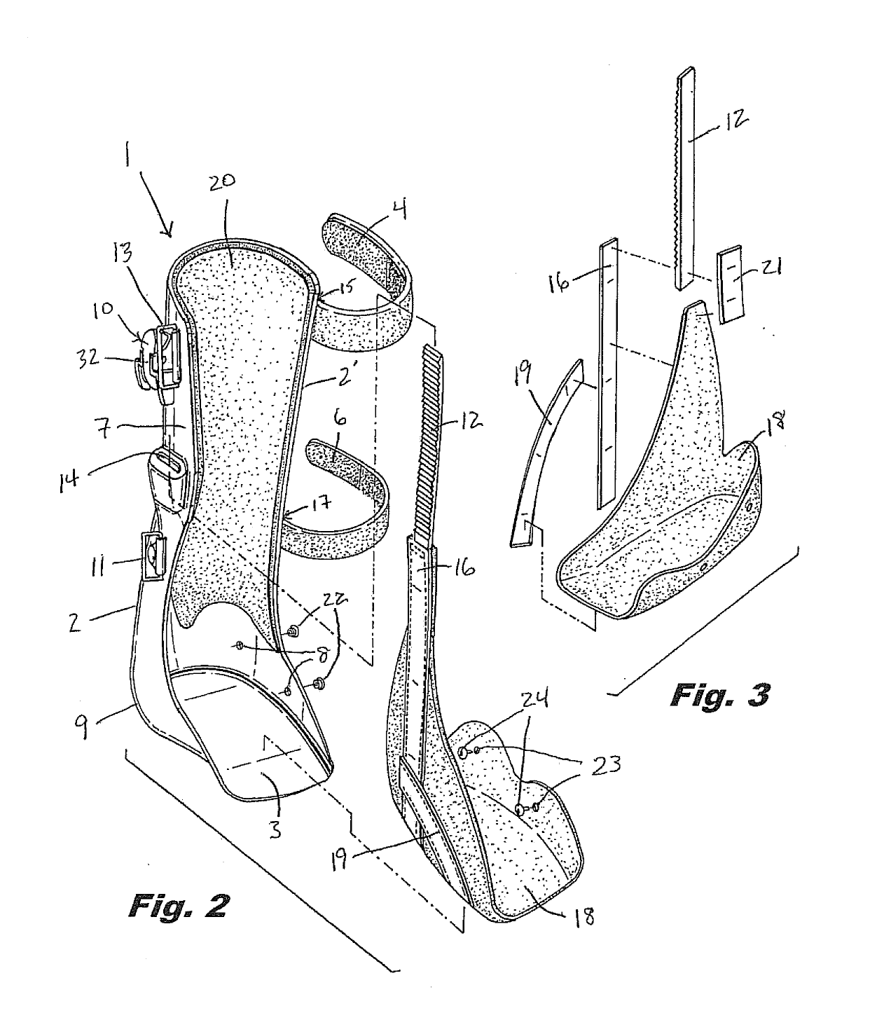

Referring first to FIG. 1, shown is a lateral side view of the molded ankle foot orthosis 1 showing one preferred exemplary embodiment of the invention constructed in accordance with this invention having two securing straps 4, 6 for securing the orthosis 1 to the ankle and lower limb of the wearer. As depicted more clearly in FIG. 2, where an exploded perspective view of one exemplary embodiment of a custom molded ankle foot orthosis 1 constructed in accordance with this invention. As will be described in more detail below orthosis 1 comprises custom molded stirrup or foot support 7, mounting or securing straps 4, 6, and an arch suspension system comprising lifting pad 18, cam buckle 10, lifting ladder strap 12 affixed to strap 16. The arch suspension system is referred to as a “medial” arch suspension system.

Preferably, the stirrup or foot support 7 is a rigid or semi-rigid member formed, e.g., molded as an integral unit, of any suitable plastic material and is shaped to accommodate the foot and lower limb of the wearer. The stirrup or foot support 7 may be a custom orthotic or may be of a more universal shape. The stirrup or foot support 7 is designed to be positioned directly under the talonavicular joint of the foot as dictated by markings on an impression cast of the wearer's foot, taken by the wearer's practitioner. The stirrup or foot support 7 includes a bottom portion 3 having a heel receiving end 9, a contiguous medial side portion 2, and a contiguous lateral side portion 2′. Preferably, stirrup or foot support 7 is lined with a cushioning material 20 for comfortability of the wearer. Preferably the cushioning material is permanently secured to the inner portion of the stirrup 7. Alternatively, such cushioning material 20 may optionally be removable to allow for washing or easy replacement.

Medial side portion 2 of foot support 7 contiguously projects upward from the bottom portion 3 on the medial side adjacent the heel receiving end 9. In a similar manner lateral side portion 2′ projects upward from the bottom portion 3 on the lateral side adjacent the heel receiving end 9. As best seen in FIGS. 1 and 2, the medial side portion 2 and lateral side portion 2′ are elongated and shaped to accommodate the medial and lateral sides of the lower leg of the wearer and are preferably molded, formed, e.g., of the same material as the bottom portion 3. In order to secure the entire stirrup or foot support 7, i.e., medial side portion 2 and lateral side portion 2′, onto the leg of the wearer plural mounting straps 4 and 6 are provided. Preferably, mounting straps 4, 6 are hook and loop straps, but may also be any other type of known securing mechanisms or straps. Mounting straps 4, 6 are preferably permanently affixed to lateral side 2′ of foot support 7 at 17, 15, respectively. In particular, the strap 4 serves to secure the top of medial and lateral side portions on the anterior of the wearer's leg, while mounting strap 6 serves to secure the bottom medial and lateral side portions on the anterior of the wearer's leg.

Mounting straps 4, 6 are preferably relatively wide straps of a soft, durable and flexible material, e.g., nylon, felt, or other material, that is arranged to be formed into a loop and extended through longitudinally extending hooks 13, 11, respectively in the medial side portion 2. Mounting straps 4, 6 are preferably permanently fixed to the lateral side portion at 17, 15. Preferably, the medial side 2 of foot support 7 comprises hooks 13, 11 for removably securing mounting straps 4, 6, respectively. Hooks 13, 11 are preferably located closely adjacent the anterior edge of the medial side portion 2. The mounting straps 4, 6 include a strip of a multi-loop component of hook and loop fastening system secured, e.g., sewn, on a surface of the straps 4, 6 adjacent one end thereof. A cooperating strip of a multi-hook component of that hook and loop fastening system is secured, e.g., sewn, on an opposite surface of mounting straps 4, 6 adjacent the other end thereof. The sides of mounting straps 4, 6 are arranged to be fed through hooks 13, 11, respectively, and brought into releasable engagement with each other to secure the straps in place and to allow for the adjustment of the tightness of the medial and lateral side portions 2, 2′, respectively, of stirrup or foot support 7 on the leg of the wearer.

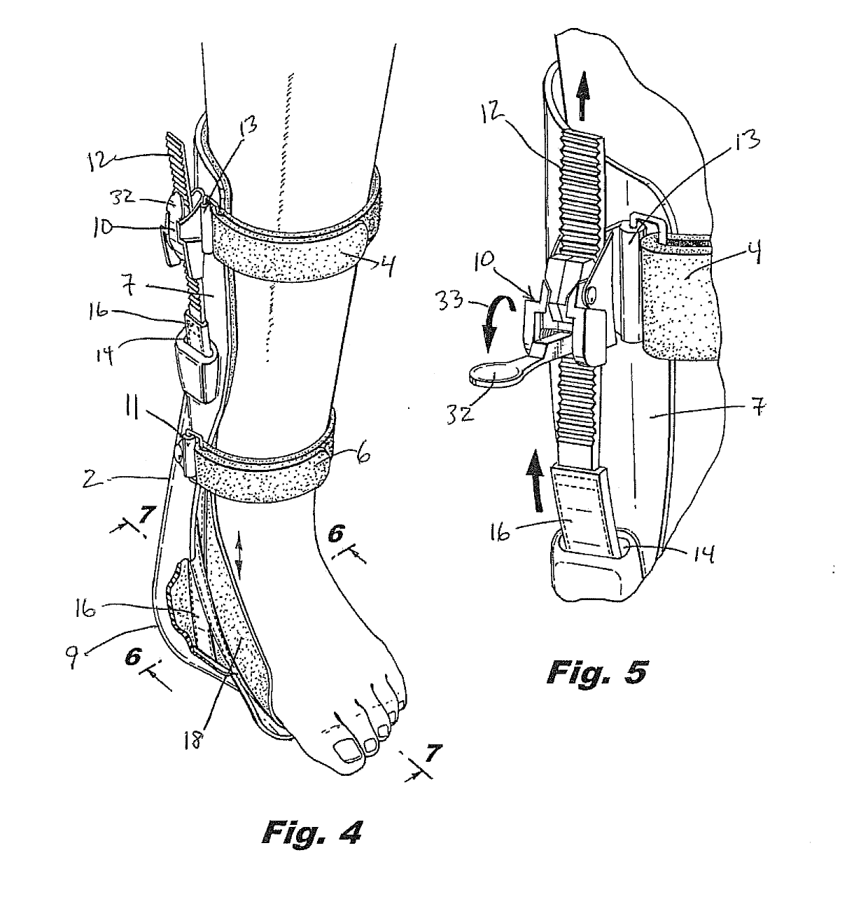

Once the wearer's foot is positioned in the stirrup or foot support 7, as shown in FIG. 1, and the medial and lateral side portions 2, 2′, respectively, are secured in place by the mounting straps 4 and 6, the adjustment of the ankle foot orthosis 1 can be accomplished to effect the lifting of selected portions of the wearer's foot on the stirrup or foot support 7. In particular, referring to FIGS. 1, 4 and 5, the foot support 7 preferably comprises an arch suspension system positioned on the inner portion of bottom portion of foot support 7, and is configured to be pulled and secured in a dorsal and lateral direction by the wearer using the earn buckle operated mechanism 10. As best seen in FIGS. 4 and 5, the arch suspension system includes cam buckle operated mechanism 10, ladder strap 12 and strap 16 positioned through aperture 14 in the medial portion 2 of stirrup or foot support 7. Strap 16 is preferably secured on its lower end to arch lifting pad 18 in the medial arch area on the inner side of medial portion 2 of stirrup or foot support 7. Strap 16 is secured, either permanently or removably, on its upper end to ladder strap 12, which is preferably fed through and engaged with cam buckle 10 in a manner such that it allows for adjustment of the lateral and distal positioning of lifting pad 18 to provide to support or suspend the talonavicular joint for full control of the hind foot/ankle complex. As shown in FIG. 5, with ladder strap 12 fed into cam buckle 10, using handle 32 through movement in an up and down motion as indicated by arrow 33 ladder strap 12 is pulled in an upward lateral motion and through strap 16 to further pull lifting pad 18 in an upward motion to provide to support or suspend the talonavicular joint for full control of the hind foot/ankle complex.

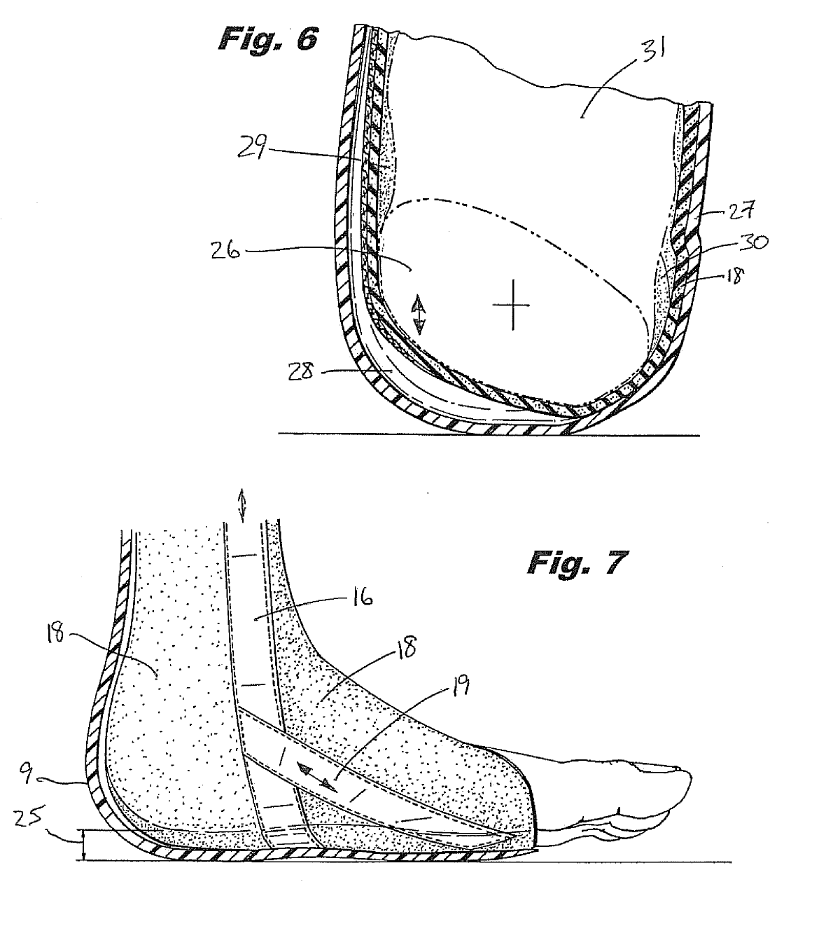

The upward motion to provide to support or suspend the talonavicular joint for full control of the hind foot/ankle complex is further depicted in FIGS. 6 and 7, which show a cross-sectional view of the molded ankle foot orthosis 7 along line 6-6 as shown in FIG. 4, and a cross-sectional view of the molded ankle foot orthosis 7 along line 7-7 as shown in FIG. 4, respectively. In particular, as seen in FIG. 6, the upward motion upon lifting pad 18 shown in FIG. 4 provides altered pressure regions 26, 31 on the bottom surface of the wearer's foot. Moreover, pockets or gaps 28, 29, 30 are formed to add further comfort or cushion to the wearer's foot during use of the orthosis 1. As further seen in FIG. 7, during use, the wearer's foot positioned inside of lifting pad 18 is lifted a certain distance 25 above the bottom surface 3 of foot support 7 which provides the support or suspension of the talonavicular joint for full control of the hind foot/ankle complex.

Further referring to FIGS. 2 and 3, best seen is the cushion or pad 20 located on the inside surface of the foot support 7. The pad 20 preferably covers the entire inner surface of the stirrup or foot support 7 and extends upward on the inside of medial and lateral portions 2, 2′. The pad 20 may be constructed of any suitable resilient or cushiony material in a single layer or ply, or multiple layers or plies of the same or different materials.

As will be appreciated by those skilled in the art from the foregoing, the arch suspension system is configured to provide support to the talonavicular joint of the wearer's foot. As is known, this is the central joint of the medial arch of the human foot. Unlike a typical arch support or foot orthotic as found on the prior art, the arch suspension system of the present invention provides an custom molded adjustable harness type suspension to selectively lift, align and support the medial arch and the talonavicular joint of the human foot. Moreover, because cam buckle 10 is configured to be releasably and adjustably connected to the lifting pad 18 via straps 12 and 16, the wearer or practitioner can adjust the tension, e.g., the amount of lift, provided by the straps 12, 16 of the arch suspension system, at the talonavicular joint. Further still, the adjustability of the connection between the strap 12 and the cam buckle 10 enables the wearer to correct or adjust the amount of lift or tension as he/she desires and to adapt to the support over a period of minutes or hours. Thus, the arch suspension system provides the wearer with the ability to implement and adjust the support of the critical area of his/her arch to achieve a new level of comfort and control not possible with standard foot orthoses or ankle foot orthoses. These advantages are achieved by providing an adjustable lifting force in a superior-lateral direction to the medial arch of the human foot.

As should also be appreciated by those skilled in the art from the foregoing, the use of the cam buckle operated adjustable custom molded arch suspension system can be used for a variety of clinical indications to provide beneficial effects, including severe pronation, mild charcot arthopathy, sinus tarsi, ankle arthritis, ankle instability, adult acquired flat foot due to PTTD (Stage II & Stage III), off-loading of mid-foot plantar ulcerations, etc.

As mentioned earlier the ankle foot orthosis 1 is merely exemplary of various braces that can be constructed in accordance with this invention. Thus an orthosis may be constructed to include the arch suspension system using other known buckles. Moreover, the stirrup's inner pad may be constructed differently than that shown and described above. Other variations are also contemplated. Moreover, in the case of a medial lifting pad, that pad could be constructed so that it provides some stretch type forgiveness, to provide an elastic type support under the talonavicular area. Also, as noted above, the stirrup or foot support could be either a custom or non-custom design. A custom stirrup or foot support can be fabricated from a plaster model made of the user's foot. The stirrup or foot support has a contoured arch and are designed to control subtalar and midtarsal joint motion of the human foot. Non-custom stirrups or foot supports could be fabricated from models of average foot shapes and sizes.

In the claims, means or step-plus-function clauses are intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that such embodiments are merely exemplary and that the invention is not limited to those precise embodiments, and that various changes, modifications, and adaptations may be effected therein by one skilled in the art without departing from the scope or spirit of the invention as defined in the appended claims. The scope of the invention, therefore, shall be defined solely by the following claims. Further, it will be apparent to those of skill in the art that numerous changes may be made in such details without departing from the spirit and the principles of the invention. It should be appreciated that the present invention is capable of being embodied in other forms without departing from its essential characteristics.

What is claimed is:

1. An apparatus for a custom molded ankle foot orthosis having an adjustable arch suspension system, said apparatus comprising:
a molded stirrup having lateral, medial and bottom portions, said stirrup configured to receive the foot of a wearer and comprising a bottom portion having a heel receiving end, a contiguous medial portion, and a contiguous lateral portion, said medial and lateral portions extending upward from said bottom portion adjacent said heel receiving end;
a medial lifting pad positioned on the inner side of said stirrup, said lifting pad fixedly secured on a first end to an inner lateral side of said stirrup and secured on a second end to a first end of a strap, said strap being positioned through an aperture on said medial portion of said stirrup and adjustably connected at a second end to an adjustment mechanism; and
at least two mounting straps arranged to secure the upper and lower lateral and medial portions of said stirrup to a wearer's leg;
wherein said adjustment mechanism engages said second end of said strap and applies a lifting force to said medial lifting pad at a wearer's medial arch joint.

2. The apparatus of claim 1, wherein the amount of lifting force provided by said lifting pad is adjustable.

3. The apparatus of claim 1, wherein said mounting straps further comprise hook and loop fastening systems.

4. The apparatus of claim 1, wherein said adjustment mechanism is a cam buckle.

5. The apparatus of claim 4, wherein said cam buckle comprises:
a frame having a pair of side walls with front and rear ends,
a loop pivotally attached to said side walls, the loop being adapted for holding one end of said strip of web-like material,
a first bar extending between said side walls and attached thereto at a position forwardly of the pivotable attachment of the loop,
a second bar mounted on the side walls and extending generally parallel to the first bar, the second bar being adapted for holding said second end of said strap, and
said buckle being movable between a locked and an unlocked position.

6. The apparatus of claim 1, wherein the outer material of said stirrup is selected from the group consisting of plastic, vinyl, composite, ethylene, polyethylene based plastic and polymer.

7. The apparatus of claim 1, wherein an inner side of said stirrup includes a cushioning material.

8. The apparatus of claim 1, wherein said stirrup has a thickness between 0.05 inches and 0.10 inches.

9. The apparatus of claim 1, wherein said stirrup is configured for use within a wearer's shoe.

10. The apparatus of claim 1, wherein said ankle foot orthosis is washable.

11. The apparatus of claim 1, wherein said ankle foot orthosis is X-ray transparent.

12. The apparatus of claim 1, wherein said ankle foot orthosis is design to treat clinical indications selected from the consisting of severe pronation, mild charcot arthopathy, sinus tarsi, ankle arthritis, ankle instability, adult acquired flat foot due to posterior tibial tendon dysfunction (PTTD) (Stage II & Stage III), and off-loading of mid-foot plantar ulcerations.
